(19) United States
(12) Patent Application Publication
(10) Pub. No.: US 2021/0292783 A1
(43) Pub. Date: Sep. 23, 2021

(54) CHO CELL LINE, CONSTRUCTION THEREOF AND RECOMBINANT PROTEIN EXPRESSION SYSTEM USING CHO CELL LINE

(71) Applicants: Xinxiang Medical University, Xinxiang (CN); Proteineasy Biological Products Research Institute (Henan) Co., Ltd., Xinxiang (CN)

(72) Inventors: Tianyun WANG, Xinxiang (CN); Wenjie RNE, Xinxiang (CN); Yanlong JIA, Xinxiang (CN); Jiangtao LU, Xinxiang (CN); Tianjun NI, Xinxiang (CN); Xiaoyin WANG, Xinxiang (CN); Xiao GUO, Xinxiang (CN); Yan LIN, Xinxiang (CN); Chong WANG, Xinxiang (CN)

(21) Appl. No.: 16/937,439

(22) Filed: Jul. 23, 2020

(30) Foreign Application Priority Data
Mar. 20, 2020 (CN) 202010200374.3

(51) Int. Cl. C12N 15/85; C12N 15/113; C12N 9/10

(52) U.S. Cl. CPC C12N 15/85; C12N 15/113; C12Y 204/02007; C12N 2310/20; C12N 9/1077

(57) ABSTRACT

The disclosure relates to genetic engineering, and more particularly to a CHO cell line, its construction and a recombinant protein expression system using the CHO cell line. Regardless of the presence screening pressure, the recombinant protein expression system constructed by the 60^{th} passage of the recombinant APRT gene-deficient CHO cell line is much higher than the normal recombinant CHO cell in the expression retention of the target protein EGFP. In the absence of G418 screening pressure, the expression level of the recombinant vitronectin in the recombinant protein expression system constructed by the 30^{th} passage of the recombinant APRT gene-deficient CHO cell is significantly higher than the expression level of the corresponding target protein in the normal recombinant CHO cells.

### REFERENCE TO AN ELECTRONIC SEQUENCE LISTING

The contents of the electronic sequence listing (Untitled ST25.txt; Size: 40,000 bytes; and Date of Creation: Aug. 16, 2020) is herein incorporated by reference in its entirety.

### CROSS-REFERENCE TO RELATED APPLICATIONS

This disclosure claims the benefit of priority from a Chinese Patent Application No. 202010200374.3, filed on Mar. 20, 2020. The content of the aforementioned application, including any intervening amendments thereto, is incorporated herein by reference in its entirety.

### TECHNICAL FIELD

The present disclosure relates to genetic engineering, and more specifically to a CHO cell line, its construction and a recombinant protein expression system using the same.

### BACKGROUND

Chinese hamster ovary (CHO) cell expression system is the most widely used mammalian cell expression system for the research, development and production of recombinant therapeutic proteins (antibodies) and other protein therapeutics. The CHO cells have many advantages. For example, the CHO cells are easy to be genetically modified, expanded and transfected and are able to perform correct protein folding and glycosylation; the CHO cells can secrete expression proteins which are easy to be purified; and the CHO cells have relatively high cell density and are suitable for suspension culture and large-scale production. In the industrial production of recombinant therapeutic proteins, the CHO cells are often required to be stably cultured for at least 30 passages during the expansion process from the working cell bank to the large-scale bioreactor. However, unstable recombinant protein expression and even significantly-reduced expression occur in the long-term culture. In addition, different recombinant CHO cell clones show diversity in transgene expression level and stability. The expression instability and high heterogeneity of phenotypes among different cell clones render the screening of recombinant CHO cell lines with stable and highly-efficient expression time- and effort-consuming and unpredictable, which causes an extension in the period of research, development and production and an increase in the cost of the cell culture, significantly limiting the industrial production of recombinant therapeutic proteins. Therefore, there is an urgent need to develop a genetic and cell engineering method to establish a high-efficiency and stable CHO cell expression system to improve the expression level and stability of the protein of interest. In this way, there is an urgent need to shorten the period of research and development and reduce the production cost, promoting the development of biopharmaceutical industry.

Adenine phosphoribosyltransferase (APRT) plays a key role in enzymatic transformation of adenine to adenosine monophosphate (AMP) in the salvage pathway of ATP synthesis in organisms. APRT gene mutant is resistant to 2,6-diaminopurine (DAP) or azaadenine, and thus can be used to screen and identify the APRT gene-deficient cells. In a medium containing alanosine, azaserine and adenine, the cells with APRT activity can still survive on the salvage pathway of adenine, while the de novo synthesis is blocked by alanosine and azaserine. So far, there is no report on constructing APRT gene-deficient cells by knocking out the APRT gene from CHO cells by CRISPR/Cas9 gene editing technique to further achieve the highly effective and stable expression of recombinant proteins.

### SUMMARY

To overcome the defects of the existing art, a first object of the disclosure is to provide an APRT gene-deficient CHO cell line by knocking out the APRT gene, which improves the expression level and the long-term expression stability when applied in the expression of recombinant proteins.

A second object of the disclosure is to provide a method for effectively constructing the above APRT gene-deficient CHO cell line.

A third object of the disclosure is to provide an application of the above APRT gene-deficient CHO cell line in the efficient and stable expression of a recombinant protein.

A fourth object of the disclosure is to provide a recombinant protein expression system, which is constructed by transfecting the APRT gene-deficient CHO cells with an expression vector carrying an APRT gene-weakened expression cassette and a target gene expression cassette, and can effectively and stably express a target protein.

For achieving the above objects, the present disclosure provides the following technical solutions.

In a first aspect, the disclosure provides a APRT gene-deficient CHO cell line, wherein the APRT gene-deficient CHO cell line is constructed by knocking out the APRT gene from a normal CHO cell line.

In an embodiment, the knockout of the APRT gene is performed by gene editing, and the APRT gene has a sequence as shown in SEQ ID NO:1.

In an embodiment, the normal CHO cell line is CHO-K1 or CHO-S.

In a second aspect, the disclosure provides a method of constructing the above-mentioned APRT gene-deficient CHO cell line, including:knocking out the APRT gene from the normal CHO cell line by CRISPR/Cas9 gene editing technique to construct the APRT gene-deficient CHO cell line.

In an embodiment, the knockout of the APRT gene comprises the following steps.

(1) designing sgRNA sequence I and sgRNA sequence II of two target sites according to the APRT gene sequence No. X03603.1 of Hamster in the GeneBank of NCBI; wherein during the knockout of the APRT gene, a sequence between the two target sites is knocked out, which results in a fragment deletion in the APRT gene, ensuring the complete loss of the functions of the APRT gene;

(2) adding a first sticky end and a second sticky end respectively to the sgRNA sequences I and II designed in step (1) to synthesize 2 pairs of primers, subjecting the two pairs of primers to annealing to correspondingly produce double-stranded DNA fragments carrying sticky ends; and respectively ligating the double-stranded DNA fragments into two CRISPR/Cas9 expression vectors respectively carrying fluorescent reporter genes I and II to construct two CRISPR/Cas9-sgRNA vectors; and

(3) co-transfecting the two CRISPR/Cas9-sgRNA vectors into the normal CHO cell line; selecting monoclonal cells containing signals of the two fluorescent reporter genes I and II by flow cytometry for expanding cultivation; and subjecting the monoclonal cells to APRT gene knockout verification through PCR amplification and sequencing to finally obtain the APRT-deficient CHO cell line.

In an embodiment, in step (1), the step of designing the sgRNA sequences I and II of the two target sites comprises:

(a) designing a pair of amplification primers to amplify a selected fragment of the APRT gene by PCR, wherein the pair of amplification primers is shown as follows:

APRT-PCR-L:
(SEQ ID NO: 2)
5′-CCAGGCTTTCAATTTGAGGT-3′
APRT-PCR-R:
(SEQ ID NO: 3);
5′-ACTCATCCAGGGTCAACGAG-3′

subjecting the amplified sequence to cloning and sequencing for verification, wherein the desired amplified sequence is shown in SEQ ID NO:4;

(b) designing the target sites of the sgRNA sequences I and II on the APRT gene with the help of an online tool to improve the gene knockout efficiency, wherein the two target sites are shown as follows:

APRTfw1:
(SEQ ID NO: 5)
5′-GCAGTCTCGGGGATCTTGTGGGG-3′
APRTfw2:
(SEQ ID NO: 6)
5′-AGTCACCTTAAGTCCACGCATGG-3′.

Since a U6 promoter is used in the sgRNA expression vector, the gene expression will be significantly up-regulated in the presence of a guanine (G) in the transcription start site. Therefore, in step (2), the forward primer of each of the two pairs of primers carrying a sticky end has a G at 5′ end thereof, and there is a cytosine (C) at the corresponding reverse primer. If the starting base at 5′ end of the forward primer is not G, it is required to additionally introduce a guanine at 5′ end of the forward primer and simultaneously introduce a cytosine (C) at the 3′ end of the corresponding reverse primer to ensure a high expression level of the gene.

In an embodiment, in step (2), two CRISPR/Cas9 expression vectors pX458-ECFP carrying the gene of fluorescent protein ECFP and pX458-DsRed2 carrying the gene of fluorescent protein DsRed2 are respectively linearized in the presence of endonuclease Bbs I, purified and recovered sequentially to obtain DNA fragments of the vectors with a sticky end. The double-stranded DNA fragments respectively synthesized from the two sgRNA sequences designed in step (1) are respectively ligated to the DNA fragments of the vectors followed by transformation and screening to obtain the CRISPR/Cas9-sgRNA expression vectors.

In an embodiment, in step (3), in order to obtain stable gene-knockout monoclonal cells, the two CRISPR/Cas9-sgRNA vectors are co-transfected into the normal CHO cells, and 72 h later, such CHO cells are sorted by flow cytometry to obtain single cells with DsRed2 and ECFP positive. The single cells with DsRed2 and ECFP positive are cultured for 14 d, and then subjected to enlarged culture and subsequent PCR amplification verification. In an embodiment, sequences of the amplification primers used in the PCR amplification are shown as APRT-PCR-L and APRT-PCR-R in step (a) of step (3). For the CHO cell line without undergoing gene knockout, the fragment amplified by such pair of primers has a length of 738 bp, so it can be deduced that the APRT gene in the CHO cell line has undergone deletion of a fragment when the amplified fragment obtained in the PCR verification has a length significantly less than 738 bp.

In a third aspect, the disclosure further provides an application of the above-mentioned APRT gene-deficient CHO cell line in the construction of a recombinant protein expression system, including:

inserting an APRT gene-weakened expression cassette into an expression vector carrying an expression cassette of a target protein gene to construct a recombinant protein expression vector;

and transfecting the recombinant protein expression vector into the APRT gene-deficient CHO cell line to construct recombinant CHO-APRT cells which can effectively and stably express a target protein.

Compared to the prior art, the disclosure has the following beneficial effects.

The APRT gene-deficient CHO cell line in the disclosure, in which the APRT gene shown in SEQ ID NO:1 is knocked out, can be used to construct a recombinant protein expression system, which significantly improves expression level and long-term expression stability of target genes in CHO cells, overcoming the defect of low stability existing in the current CHO cell expression system.

When using the APRT gene-deficient CHO cell line provided herein to construct a recombinant protein expression system for expressing recombinant proteins, regardless of the presence of the screening pressure, the expression retention of target protein EGFP in the 60^{th} passage of the recombinant APRT gene-deficient CHO cell line is higher than 100% (compared with that of the 1^{st} passage of the recombinant cell), and is much higher than that in the normal recombinant CHO cells (lower than 57%). In the absence of G418 screening pressure, the expression level of the vitronectin (314.22±25.14 ng/mL) in the 30^{th} passage of the recombinant APRT gene-deficient CHO cell is significantly higher than the expression level (239.14±19.13 ng/mL) of corresponding target protein in the normal recombinant CHO cells.

### BRIEF DESCRIPTION OF THE DRAWINGS

FIG. 1 shows the screening of an APRT gene-knockout CHO monoclonal cell line by PCR amplification according to Example 1 of the disclosure.

FIG. 2 shows the detection of the proliferation of APRT gene-deficient CHO monoclonal cell lines and normal CHO cells using CCK-8 assay according to Example 2 of the disclosure.

FIG. 3 shows the eukaryotic expression vector pWTY3G-EGFP driven by an EF-1α promoter.

FIG. 4 shows the detection of the relative level of APRT gene copy number in the recombinant CHO cells by real-time quantitative PCR (qPCR) according to Example 3 of the disclosure, where the recombinant CHO cells are obtained by transfecting APRT gene-deficient CHO cells and normal CHO cells both with an expression vector without an APRT gene-weakened expression cassette (normal vector) or an expression vector with an APRT gene-weakened expression cassette (weakened vector).

FIG. 5 shows the fluorescence expression of EGFP in the recombinant CHO cells according to Example 3 of the disclosure, where the recombinant CHO cells are obtained by transfecting APRT gene-deficient CHO cells and normal CHO cells both with the expression vector without the APRT gene-weakened expression cassette (normal vector) or the expression vector with the APRT gene-weakened expression cassette (weakened vector).

FIG. 6 shows the expression level of EGFP in the recombinant CHO cells according to Example 3 of the disclosure, where the recombinant CHO cells are obtained by transfecting APRT gene-deficient CHO cells and normal CHO cells both with the expression vector without the APRT gene-weakened expression cassette (normal vector) or the expression vector with the APRT gene-weakened expression cassette (weakened vector).

FIG. 7 shows the expression retention of target protein EGFP in the 60^{th} passage of the recombinant APRT-deficient CHO cells and normal CHO cells according to Example 3 of the disclosure.

### DETAILED DESCRIPTION OF EMBODIMENTS

This disclosure will be further illustrated below with reference to the embodiments. Unless otherwise specified, instruments and reagents used in the following experimental examples are all commercially available. The CHO cells in the following examples are the commercially-available CHO-K1 cells.

### Example 1 Construction of APRT Gene-Deficient CHO Cell Line

Provided herein was a method of preparing the APRT gene-deficient CHO cell line, which was specifically described as follows.

1. Determination of Target Sites for a Candidate Gene

(1) Amplification of Partial Sequence of APRT Gene

Primers for amplification were designed according to the sequence of APRT gene (No. X03603.1, SEQ ID NO:1) recorded in GenBank of NCBI, and were shown as follows:

APRT-PCR-L:
(SEQ ID NO: 2)
5′-CCAGGCTTTCAATTTGAGGT-3′;
and
APRT-PCR-R:
(SEQ ID NO: 3)
5′-ACTCATCCAGGGTCAACGAG-3′.

The APRT gene fragment was amplified by PCR, and the amplified product was cloned and sequenced for verification. The desired amplified sequence was shown in SEQ ID NO:4.

(2) Determination of Target Sites of sgRNA Sequences

The target sites of sgRNA sequences on the APRT gene were designed with the help of an online tool (http://crispr.mit.edu/), and were shown as follows:

APRTfw1:
(SEQ ID NO: 5)
5′-GCAGTCTCGGGGATCTTGTGGGG-3′;
and
APRTfw2:
(SEQ ID NO: 6)
5′-AGTCACCTTAAGTCCACGCATGG-3′.

2. Construction of a sgRNA Expression Vector

(1) Designing and Synthesis of Primers

Two pairs of primers were designed and synthesized according to the above target sites of sgRNA sequences, and respectively added with a sticky end at the 5′ end;

It should be noted that since a U6 promoter was used in the sgRNA expression vector, the gene expression will be significantly up-regulated in the presence of a guanine (G) in the starting site of the gene transcription. Therefore, during the designing process of the primers, if the starting base at 5′ end of the forward primer was not G, it was required to additionally add a guanine to ensure a high expression level. In this case, a cytosine (C) was required to be added at the 3′ end of the corresponding reverse primer;

(2) Preparation of Double-Stranded DNA Fragments by Annealing

The two pairs of primers synthesized in step (1) were respectively subjected to annealing to produce double-stranded DNA fragments both with a sticky end. Specifically, the two pairs of primers were respectively phosphorylated and then transferred to a PCR instrument for denaturation and annealing, where the phosphorylation was performed through the steps of: mixing 1.0 μL of respective primers (100 μM), 1.0 μL of 10×T4 Ligation Buffer (NEB), 0.5 μL of T4 Polynucleotide Kinase (NEB M0201S) and 6.5 μL of ddH₂O uniformly to produce a phosphorylation system (10.0 μL); and incubating the phosphorylation system at 37° C. for 30 min to complete the phosphorylation; the denaturation was performed at 95° C. for 5 min; and the annealing was performed by reducing the temperature from 95° C. to 25° C. at 5° C./min;

(3) Linearization of CRISPR/Cas9 Expression Vectors

Two CRISPR/Cas9 expression vectors pX458-ECFP carrying the gene of fluorescent protein ECFP and pX458-DsRed2 carrying the gene of fluorescent protein DsRed2 were linearized in the presence of endonuclease Bbs I, purified and recovered to obtain DNA fragments with a sticky end, where the digestion was performed through the steps of: mixing 1.0 μg of the vector pX458-DsRed2 or pX458-ECFP, 3.0 μL of 10×NEB Buffer 2.1 and 1.0 μL of Bbs I (NEB) followed by addition of ddH₂O to a volume of 30.0 μL; and incubating the system at 37° C. for 2 h to complete the digestion; and the digested product was purified using a QIAquick PCR Purification Kit and dissolved with 30.0 μL of ddH₂O for recovery.

(4) Construction of sgRNA Expression Vectors.

CRISPR/Cas9 expression vectors containing sgRNA were obtained by ligation, transformation and screening, where the ligation was performed through the steps of mixing 0.5 μL of the double-stranded DNA fragment with the sticky end obtained in step (2), 2.0 μL of the vector DNA with the same sticky end obtained in step (3), 0.5 μL of T4 DNA ligase (NEB M0202S) and 1.0 μL of 10×T4 Ligation Buffer (NEB) followed by addition of ddH₂O to a volume of 10.0 μL to produce a ligation system; and reacting the ligation system for 1 h to complete the ligation; the transformation and screening were performed through the steps of transforming the ligated product into E. coli DH5a cells; spreading the cells on an ampicillin-resistant plate; incubating the plate at 37° C. overnight; and picking up a single colony for sequencing verification to obtain the expression vectors pX458-aprt-1 and pX458-aprt-2 respectively capable of expressing ECFP and DsRed2.

3. Transfection of CHO Cells, and Screening and Identification of Gene-Knockout Monoclonal Cell Line

The pX458-aprt-1 and pX458-aprt-2 expression vectors were mixed in equal weight and transfected into CHO-S cells in a liposome-mediated manner. Then the CHO cells were subjected to screening and verification to obtain APRT gene-knockout monoclonal cell line, which was specifically described as follows.

(1) CHO-S cells were cultured in a DMEM-F12 medium containing 10% inactivated fetal bovine serum at 37° C. and 5% CO₂. Before the transfection, 2.0×10⁵ CHO-S cells were seeded in a 24-well culture plate and cultured for 24 h. When the confluency reached about 90%, the cells were used for the transfection.

(2) 1.5 μg of pX458-aprt-1 and 1.5 μg of pX458-aprt-2 vectors were diluted with 150.0 μL of a reduced serum media (Opti-MEM). 0.75 μL of a liposome Lipofectamine 3000 was diluted with 150.0 μL of the reduced serum media (Opti-MEM) and added to the diluted expression vector DNA solution. Then the reaction mixture was fully mixed and incubated at room temperature for 20 min.

(3) The medium in the 24-well plate in step (1) was discarded, and then the CHO-160 cells in respective wells were added with 300 μL of the mixture of the plasmid DNA and the liposome incubated in step (2). Another three wells were treated in the same manner and used as parallel controls, and the wells in which the cells were not added with the transfection mixture were used as negative control. All cells were cultured at 37° C. and 5% CO₂ for 1.5 h, and then the medium was replaced for continuous culture;

(4) In order to obtain stable gene-knockout monoclonal CHO-S cells, monoclonal cells with double positive of DsRed2 and ECFP were sorted by flow cytometry into a 96-well plate containing 150 μL of fresh medium after 72 h of the transfection, and cultured for 14 d. After that, the monoclonal CHO-S cells were transferred to a 48-well plate for enlarged culture and subsequent PCR verification and analysis, in which those monoclonal cells passing the verification and analysis were the successfully constructed APRT-deficient CHO cells.

The PCR verification and analysis was performed as follows.

(a) Extraction of Genomic DNA from Monoclonal Cells

A small number of cells (about 1×10⁶-10⁷) were collected and centrifuged at 350×g for 5 min. The supernatant was discarded, and the pellet was added with 20.0 μL of a cell lysis solution containing 100 mM KCl, 20 mM Tris-HCl (pH 9.0), 0.3% Triton X-100 and 1.0 mg/mL proteinase K, gently mixed using a pipette and incubated at 55° C. for 15 min for complete lysis. Then the system was incubated at 95° C. for 10 min to denature the proteinase K, and the resulting lysate containing the genomic DNA of the cells can be used as a PCR template and was stored at −20° C. for use.

(b) Primers APRT-PCR-L (SEQ ID NO:2) and APRT-PCR-R (SEQ ID NO:3) were used to amplify the target fragment containing the target sites by PCR, and the amplified products were analyzed to determine whether the base deletion occurred in the monoclonal cell lines.

Results of the agarose gel electrophoresis detection for PCR products in this example were shown in FIG. 1, where WT indicated positive plasmid control; NC indicated blank negative control; and M indicated DNA molecular weight marker. It can be seen from FIG. 1 that single-cell clones 8 and 14 were homozygotes with deletion of a fragment. The single-cell clones 8 and 14 were further identified by sequencing analysis to be APRT gene-knockout cell lines. Then these APRT gene-knockout monoclonal cell lines were subjected to enlarged culture and cryopreserved in liquid nitrogen.

### Example 2 Identification of Biological Characteristics of APRT-Deficient CHO Cells Obtained in Example 1

1. Whether the gene-deficient cell line can perform normal growth and passage was demonstrated by examining biological characteristics of the cells such as cell proliferation and doubling time. Wild-type CHO-S cells were used as control to verify the growth characteristics of the APRT-deficient CHO monoclonal cells obtained in Example 1, where the verification included observation of cell morphology and growth status and detection of cell proliferation by CCK-8 assay, and was performed to determine whether the APRT gene-deficient CHO cell line can perform normal growth and passage.

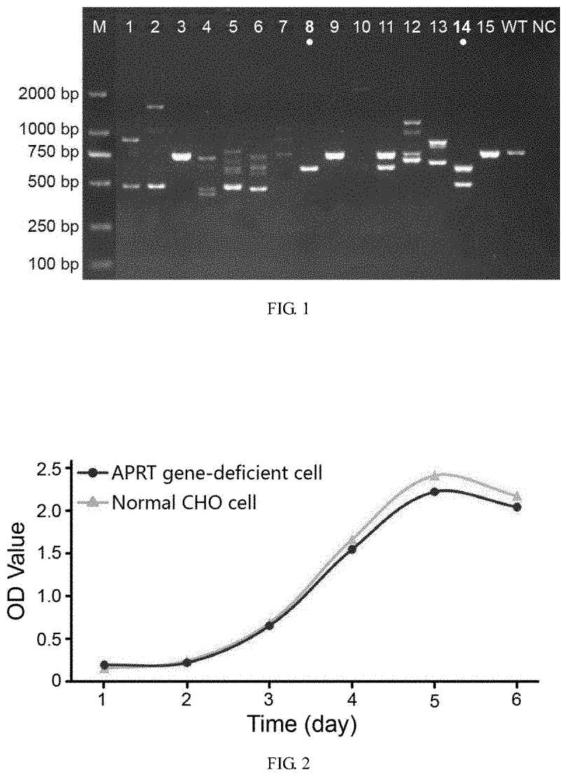

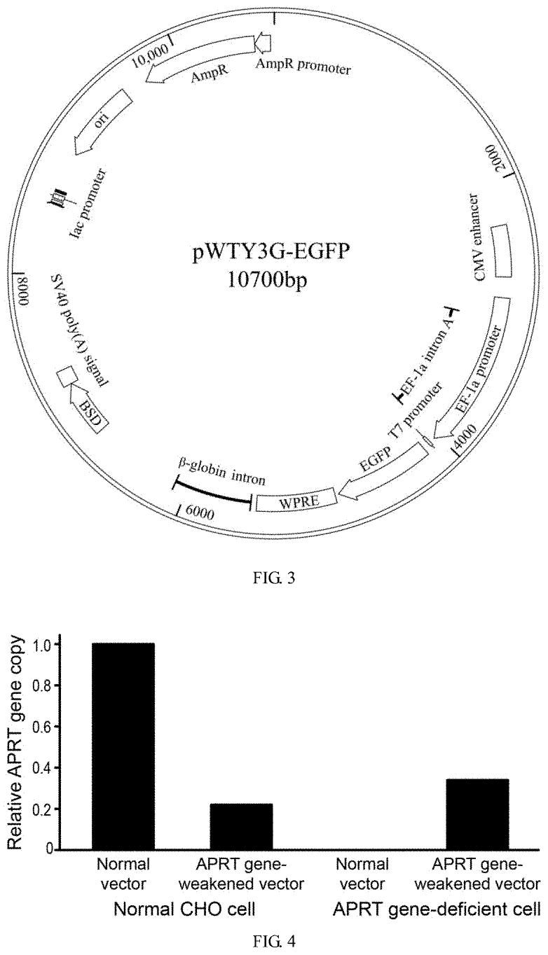

2. A Cell Counting Kit-8 (CCK-8 detection method, Beyotime Biotechnology Co., Ltd.) was employed to detect the proliferation status of cells, and the detection results were shown in FIG. 2;

It can be seen from the results that there was no significant difference between the APRT-deficient CHO cell line and the normal CHO cells in the biological characteristics such as growth status, morphology, proliferation and doubling time, indicating that the APRT-deficient CHO cell line had normal abilities to grow, proliferate and passage.

### Example 3 Application of the APRT Gene-Deficient CHO Cell Line in Example 1 in the Construction of Expression System of Target Gene EGFP and Expression Analysis of Target Gene

1. Construction of APRT Gene-Weakened Vector pWTY3G-APRT-EGFP-Mut

An eukaryotic expression vector pIRESneo2 (Clontech Co., Ltd.) was used as a base vector to construct an eukaryotic expression vector pWTY3G-EGFP in which the expression of enhanced green fluorescent protein (EGFP) was driven by an EF-1α promoter, and the sequence of the vector pWTY3G-EGFP was shown in SEQ ID NO:7. An APRT gene expression cassette carrying mutated start codon and driven by a weaker SV40 promoter was synthesized (SEQ ID NO:8) and inserted upstream of the EF-1α promoter of the base vector to construct an eukaryotic expression vector named pWTY3G-APRT-EGFP-mut which can simultaneously express EGFP and weakened APRT, and had a sequence as shown in SEQ ID NO:9.

2. Transfection of CHO Cells

The APRT gene-deficient CHO-S cells constructed in Example 1 and normal CHO-S cells were used herein for comparison. These two types of CHO cells were cultured, and inoculated into a fresh DMEM medium containing 10% inactivated fetal bovine serum at a density of 2×10⁵ cells one day before the transfection. When the conflency reached 90%, Lipofectamine 3000 (Invitrogen, USA) was used to perform the transfection, where the plasmids used for the two types of CHO cells in the transfection were the control vector pWTY3G-EGFP without the APRT gene-weakened expression cassette and the APRT gene-weakened vector pWTY3G-APRT-EGFP-mut obtained in step (1). The transfection was performed in three parallel replicates. After the transfection, cells were screened, and the relative level of APRT gene copy number in each group of recombinant CHO cells was shown in FIG. 4.

3. Observation of Transient Expression of Transfected Cell Lines

48 h after the transfection of the two groups of cells with the two types of plasmids in step (2), the transient expression of EGFP in the two groups of cells was observed by an inverted fluorescence microscope.

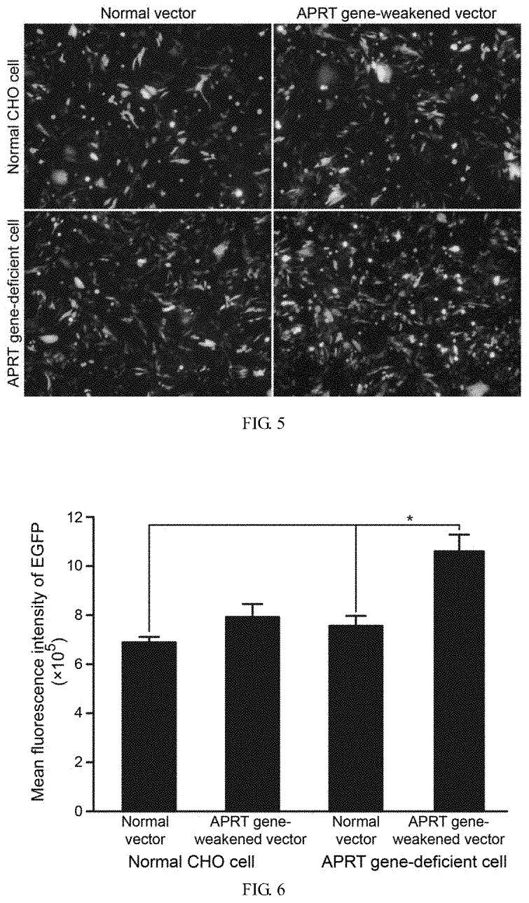

It can be concluded from the results that the two types of expression vectors had comparable transfection efficiency in the APRT-deficient and normal CHO-S cells, and there was no significant difference in the ratio of the EGFP-positive cells between the two groups of cells (shown in FIG. 5), which indicated that the knockout of APRT gene showed no significant effect on the transfection efficiency of cells. The stably-transfected 1^{st} passage of the recombinant CHO cell pools were obtained by screening, in which the EGFP expression level in the APRT-knockout cells with the APRT gene-weakened expression vector was significantly higher than that in the normal control cells and the gene-knockout control cells (shown in FIG. 6).

4. Screening of a Polyclonal CHO Cell Line with Stable Expression and Analysis of Long-Term Stable Expression of EGFP

The cells were screened in the presence of G418, and stably-transfected recombinant polyclonal cell pools were obtained after two weeks of screening. Then the cell pools were cultured to passage 60 respectively in the presence (G418⁺) and absence (G418⁻) of G418, and 10⁶ CHO cells from respective groups were analyzed by flow cytometry to measure the mean fluorescence intensity (MFI) of EGFP.

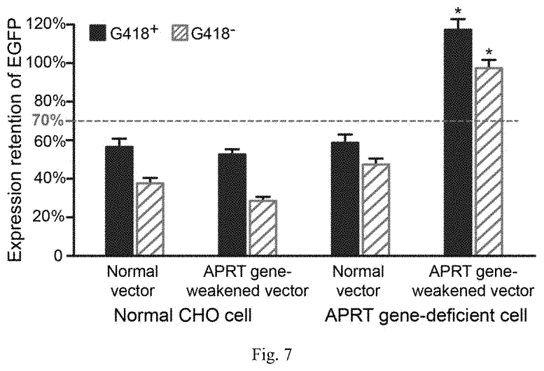

It can be seen from the results that according to the criterion for evaluating the expression stability (whether the expression level of EGFP in recombinant CHO cells of the 60^{th} passage was greater than 70% of that in the 1^{st} passage of the recombinant CHO cells), regardless of the presence of G418 screening pressure, the expression stability of EGFP in the recombinant APRT gene-deficient CHO cells transfected with the APRT-weakened expression vector of the 60^{th} passage was significantly higher that in the normal CHO cells (see FIG. 7, p<0.05).

### Example 4 Construction of Vitronectin (VTN) Expression System Using the APRT Gene-Deficient CHO Cells Obtained in Example 1 and Expression Analysis of Target Gene

The APRT gene-weakened expression vector pWTY3G-APRT-EGFP-mut constructed in Example 3 was used herein as base vector, and then the EGFP sequence of the base vector was substituted by the VTN sequence (SEQ ID NO:10) to construct an APRT-weakened expression vector pWTY3G-AP/Vitin-M capable of expressing VTN. A vector pWTY3G-VTN which was free of the APRT-weakened expression cassette and only expressed VTN was used as control.

The APRT-deficient cells and normal CHO cells were respectively transfected with the constructed expression vectors pWTY3G-AP/Vitin-M and pWTY3G-VTN. The transfected cells were cultured in a medium containing G418 (800 μg/mL) for two weeks to screen stably-transfected recombinant cell pools. The two groups of recombinant CHO cells were passaged every other 3 days and cultured to passage 30. Then the recombinant CHO cells were cultured in a 125 mL shake flask containing 30 mL of protein-free, serum-free and chemically-defined CD CHO culture medium (Life Technologies Co. Ltd., containing 8 mM of L-glutamine) for 6 days to the cell number of 1.5×10⁷. During the culture, cells were collected everyday and determined using Countstar® BioTech cell counter (Shanghai Ruiyu biotechnology Co. Ltd.) for cell density and viability. On the sixth day, the supernatant was collected by centrifugation and used in ELISA analysis of the expression of recombinant VTN.

It can be seen from the results that in the absence of G418 screening pressure, the expression level of the recombinant vitronectin (VTN) (314.22±25.14 ng/mL) in the recombinant protein expression system constructed by the APRT gene-deficient CHO cell in Example 1 of the 30^{th} passage was significantly higher than the expression level (239.14±19.13 ng/mL) of corresponding target protein in the normal CHO cells, and the expression level difference was statistically significant (p<0.05). The results indicated that the APRT gene-deficient CHO cells constructed herein could significantly improve the expression level and expression stability of the recombinant VTN.

Described above are merely preferred embodiments of the application, which are merely illustrative of the concept and features of the invention and are not intended to limit the application. Any changes, replacements and modifications made without departing from the spirit of the application should fall within the scope of the application.

What is claimed is:

1. An adenine phosphoribosyltransferase (APRT) gene-deficient CHO cell line, wherein the APRT gene-deficient CHO cell line is constructed by knocking out the APRT gene from a normal CHO cell line.

2. The APRT gene-deficient CHO cell line of claim 1, wherein the knockout of the APRT gene is performed by gene editing, and the APRT gene has a sequence as shown in SEQ ID NO:1.

3. The APRT gene-deficient CHO cell line of claim 1, wherein the normal CHO cell line is CHO-K1 or CHO-S.

4. The APRT gene-deficient CHO cell line of claim 2, wherein the normal CHO cell line is CHO-K1 or CHO-S.

5. A method of constructing the APRT gene-deficient CHO cell line of claim 2, comprising:
knocking out the APRT gene from the normal CHO cell line by CRISPR/Cas9 gene editing technique to construct the APRT gene-deficient CHO cell line.

6. The method of claim 5, wherein the knockout of the APRT gene comprises the steps of:
(1) designing sgRNA sequence I and sgRNA sequence II of two target sites according to the APRT gene sequence No. X03603.1 of Hamster in the GeneBank of NCBI;
(2) adding a first sticky end and a second sticky end respectively to the sgRNA sequences I and II designed in step (1) to synthesize 2 pairs of primers, subjecting the 2 pairs of primers to annealing to correspondingly produce double-stranded DNA fragments; and respectively ligating the double-stranded DNA fragments into two CRISPR/Cas9 expression vectors respectively carrying fluorescent reporter genes I and II to construct two CRISPR/Cas9-sgRNA vectors; and
(3) co-transfecting the two CRISPR/Cas9-sgRNA vectors into the normal CHO cell line; selecting monoclonal cells containing signals of the fluorescent reporter genes I and II by flow cytometry for culture; and subjecting the monoclonal cells to APRT gene knockout verification through PCR amplification and sequencing to obtain the APRT-deficient CHO cell line.

7. The method of claim 6, wherein step (1) comprises steps of:
(a) designing a pair of amplification primers to amplify a selected fragment of the APRT gene by PCR, wherein the pair of amplification primers is shown as follows:
APRT-PCR-L:
(SEQ ID NO: 2)
5′-CCAGGCTTTCAATTTGAGGT-3′
APRT-PCR-R:
(SEQ ID NO: 3)
5′-ACTCATCCAGGGTCAACGAG-3′;
subjecting the amplified sequence to cloning and sequencing for verification, wherein the desired amplified sequence is shown in SEQ ID NO:4; and
(b) designing the target sites of the sgRNA sequences I and II on the APRT gene with the help of an online tool, wherein the two target sites are shown as follows:
APRTfw1:
(SEQ ID NO: 5)
5′-GCAGTCTCGGGGATCTTGTGGGG-3′
APRTfw2:
(SEQ ID NO: 6)
5′-AGTCACCTTAAGTCCACGCATGG-3′.

8. The method of claim 6, wherein in step (2), a forward primer in each of the two pairs of primers has a base G at 5′ end thereof, and a reverse primer in each of the two pairs of primers has a base C at 3′ end thereof.

9. The method of claim 6, wherein in step (3), the knockout verification comprises the steps of:
extracting DNA from the monoclonal cells;
subjecting the obtained DNA to PCR amplification; and
determining a length of the amplified fragment; wherein primers used in the PCR amplification are shown as follows:
APRT-PCR-L:
(SEQ ID NO: 2)
5′-CCAGGCTTTCAATTTGAGGT-3′
APRT-PCR-R:
(SEQ ID NO: 3)
5′-ACTCATCCAGGGTCAACGAG-3′.

10. A method for constructing a recombinant protein expression system using the APRT gene-deficient CHO cell line of claim 1, comprising:
inserting an APRT gene-weakened expression cassette into an expression vector carrying an expression cassette of a target protein gene to construct a recombinant protein expression vector; and
transfecting the recombinant protein expression vector into the APRT gene-deficient CHO cell line to construct the recombinant protein expression system.

11. The method of claim 10, wherein the knockout of the APRT gene is performed by gene editing, and the APRT gene has a sequence as shown in SEQ ID NO:1.

12. The method of claim 10, wherein the normal CHO cell line is CHO-K1 or CHO-S.

13. The method of claim 11, wherein the normal CHO cell line is CHO-K1 or CHO-S.


SEQUENCE LISTING (10 sequences)

SEQ ID NO: 1 - DNA, 3960 bp, Artificial Sequence
  synthetic
ggatccggacaacacccacaccggcccctccaggtccagaaagctggccctgcgagaagcgggactgaaaaggcgtgcgggagccagaaatccaaaagggtgccaaggcatgcgtcctttttccacccagaaataaccccaggctttcaatttgaggttatttcaatatccagcaaatgcgttacttcctgccaaaagccagcctccccgcaacccactctcccagaggccccgccccgtcccgccccctcccggcctctcctcgtgctggatcgctccctaaggacgccccgctccagaagccccacctaccaaggacgccccacccttgttcccggactggtatgaccccagcctgctgacatccctccgccctttctcgtgcacgcggctatggcggaatctgagttgcagctggtggcgcagcgatccgcagtttccccgacttccccatccccggcgtgctgtttaggtgagatcacgagccagcaaggcgttggagccctgttcctgggctcccggcgaggcgcatgggcagtctcggggatcttgtggggtctccgcccccctttccccggccaccagcctctccttgttcccagggatatctcgcccctcctgaaggaccccgcctccttccgagcttccatccgcctcctggccagtcaccttaagtccacgcatggcggcaagatcgactacatcgcaggcgagtggccatgccaggccgtgctggtcccccactgtgcaggctcccctcccttcccttatgtcaccctcagtccatcccacacccatcccttctttcttcaacccctaatcctcttttagcactctgtttctcttgccttggtccctcgttgaccctggatgagtactgcgtctcccacccctgctaggtctctgacctccgccctcagtgcctgttctactagagatgaactctgctcggtccttgttcagggccagcccttctctcttggggtgtgaagttggctagcagcctggaggcaggactgtaagaagcatacgtgtgctttgagaactttgaggaaggccctggaacctccttgctaggagtagcacctaagatgaactagatgctaaaaaatgctgtatctttggggcacacgagggcatgcctgggcaggcttagagcctggtagtctcaggggctgcaccaaagtgtaattcttgtgctaaataactttcacttaccagtgccaagcacgggcttcagaaacaccctagggtcgctgaatgtccaccaggggagtcagacatgtccagagggtgagaaccccagagaattcggtagccctgacatgtgctacaattactgatgcccacttcctactggttcctcctggccatacctcaggaattagggcatgctttctgcctgctacagtagctcatcctccctggaagtgaccccagacatataccctgaactgtaaccgataaagtgcgcctgggcagatgtatttgagaggtggcaaaagtaaaccataggtgtccccgagctagatacagaaggcagataacatccccaaggctaagctgctgccccaatagccatcagccttctagttatagctagtaagacctagtattcctggtcaatactattcactcaatccttacacctcagccctaacacgccccctctctcatcctaacaggcctagactccaggggattcttgtttggcccctccctagctcaggagctgggcctgggctgtgtgctcatccggaagcgagggaagctgccaggccccacagtgtcagcctcctatgctctcgagtatggcaaggtaagcaggcagtgggtagctgtctaggagtaaatgtgggggctcagagaggttaagtcatcaggccaggtttataccaccaggaaacatggagaagctaggggtggtggtcacttgttagctactagactctcactctacttcctgtctgcaggctgaactagaaatccagaaagacgccttagaacctggccagaaagtggttgttgtagatgatctcctggccactggaggtaagagccactctgtagcataaagaggtttcaaagggataagccctatccggggtgctgactaagcaagagccttactacctgtgtctttcctcgtcccttcaccccaggaaccatgtgcgctgcctgtgagctgctgggccagctacaggctgaggtggtggagtgtgtgagcctggtggagctgacctcacttaagggcagagagaagctaggatcagtaccattcttctctctcctgcaatatgagtgactggaatggttgctgcatccctgccctcagcatctacggttgaacagtggctcagccttaccaaagtgacctttgtgagccaccagctgcactttctctaactctattcactcatttctttggtcagctgatggccatgcctatggaccacctgggtccttgcattttatatatgtgctgagtcctggagcagagcagagctactgtgggttatgacacagcagatcaataaatagtttggtacatatggtgcttcctgttgtcttgttgcatggatcacgttccatagctgccaggtgacagtccctgtcccttgggattccctgtgcaaatgttctccacagagtcaatcccacacactgccccggccgtagggtcttatagaaccagaatccgttttaagcaaaaggctttcatcagtagtctctagagtgacatgatgggaacaggggctcagtgtcaccttgcagacctcttcaaaacaggtggaaaccagtaggcacagctctgtatagatttcacagcctctaggaagttttctgggccattcctttaggaccaaaatggagagagtagcccaagggaataccttattcatattttgttgaacatcaataccaggttgtttgtagggatgctgagccacacccccagcaagactgttcccatccagacttttactggcctggtcttgctagccatagaagaaaacattgcaacaacagcagggcatcatttataggtattttatggtgacccattcagtttaatttctgtattacttttatttaaatcttacgtgactttattaaaagccaagattaacagtcacatccaaggtacagaaggttaaataaaaattttgatctcaaaacctatagttatttccattatatacagaaggcccaatttcactcttgccattcagaactctctgtccttatgaaagctggccagaagcccaagttctctgtgtgggtcttaaggctctaagcaagacagcagctttcatgagcatggagaacaggctaggctttggcctaaggctcccaactggaccttgccagtacctagatgagtagtttgtgggacgggcatctaacatactctgctctccaagtctacttccccaacacccacacaagtgctgggtctggggtacccaaagcctcaggaaccaatttagcactttatgggcacaattgctgcccatactggcagtaagcccttgggcctgtgacactgcgagggatgctcaagtgcattgtactgataaagggaacagactatcaggtaaagaaatgagtcacaacccctcagtgtggatctggtgggtcagcctttcagtgaggccagctaagtcagcagctttgtccagcttgatgtaggtgtccatgcgaatccgatgcaggctcagccagtctggcagcaactcagataggagcaacacatgtttctccatctcccctgtggaagtgcattgtggttaactgaagcacagtgcctggatgacttgcttaactcacttacccagcctggtgccttacacccctcaaggcttaacctgtgtgcagtggatccaagcagagcctgggccctgctctcaaacaccagggggatgaacagcaagggcaagacacatatacgccaagctt

SEQ ID NO: 2 - DNA, 20 bp, Artificial Sequence
  synthetic
ccaggctttcaatttgaggt

SEQ ID NO: 3 - DNA, 20 bp, Artificial Sequence
  synthetic
actcatccagggtcaacgag

SEQ ID NO: 4 - DNA, 738 bp, Artificial Sequence
  synthetic
ccaggctttcaatttgaggttatttcaatatccagcaaatgcgttacttcctgccaaaagccagcctccccgcaacccactctcccagaggccccgccccgtcccgccccctcccggcctctcctcgtgctggatcgctccctaaggacgccccgctccagaagccccacctaccaaggacgccccacccttgttcccggactggtatgaccccagcctgctgacatccctccgccctttctcgtgcacgcggctatggcggaatctgagttgcagctggtggcgcagcgatccgcagtttccccgacttccccatccccggcgtgctgtttaggtgagatcacgagccagcaaggcgttggagccctgttcctgggctcccggcgaggcgcatgggcagtctcggggatcttgtggggtctccgcccccctttccccggccaccagcctctccttgttcccagggatatctcgcccctcctgaaggaccccgcctccttccgagcttccatccgcctcctggccagtcaccttaagtccacgcatggcggcaagatcgactacatcgcaggcgagtggccatgccaggccgtgctggtcccccactgtgcaggctcccctcccttcccttatgtcaccctcagtccatcccacacccatcccttctttcttcaacccctaatcctcttttagcactctgtttctcttgccttggtccctcgttgaccctggatgagt

SEQ ID NO: 5 - DNA, 23 bp, Artificial Sequence
  synthetic
gcagtctcggggatcttgtgggg

SEQ ID NO: 6 - DNA, 23 bp, Artificial Sequence
  synthetic
agtcaccttaagtccacgcatgg

SEQ ID NO: 7 - DNA, 10700 bp, Artificial Sequence
  synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgaaggtgggtggatcacccgaggtcaggagttcaagaccagcctggccaacatggtaaaacctcgtctctactaaaaaatacgaaaaattagctggttgtggtggtgcgtgcttgtaatcccagctactcgggaggctgaggcaggagaatcacttgaatctgggaggcagaggttgcagtgagctgagatagtgccattgcactccagcctgggcaacagacggagactctgtctccaaaaaaaaaaaaaaaaatcttagaggacaagaatggctctctcaaacttttgaagaaagaataaataaattatgcagttctagaagaagtaatggggatataggtgcagctcatgatgaggaagacttagcttaactttcataatgcatctgtctggcctaagacgtggtgagctttttatgtctgaaaacattccaatatagaatgataataataatcacttctgaccccccttttttttcctctccctagactgtgaagcagaaaccccatatttttcttagggaagtggctacgcactttgtatttatattaacaactaccttatcaggaaattcatattgttgcccttttatggatggggaaactggacaagtgacagagcaaaatccaaacacagctggggatttccctcttttagatgatgattttaaaagaatgctgccagagagattcttgcagtgttggaggacatatatgacctttaagatattttccagctcagagatgctatgaatgtatcctgagtgcatggatggacctcagttttgcagattctgtagcttatacaatttggtggttttctttagaagaaaataacacatttataaatattaaaataggcccaagaccttacaagggcattcatacaaatgagaggctctgaagtttgagtttgttcactttctagttaattatctcctgcctgtttgtcataaatgcgtttagtagggagctgctaatgacaggttcctccaacagagtgtggaagaaggagatgacagctggcttcccctctgggacagcctcagagctagtggggaaactatgttagcagagtgatgcagtgaccaagaaaatagcactaggagaaagctggtccatgagcagctggtgagaaaaggggtggtaatcatgtatgccctttcctgttttattttttattgggtttccttttgcctctcaattccttctgacaatacaaaatgttggttggaacatggagcacctggaagtctggttcattttctctcagtctcttgatgttctctcgggttcactgcctattgttctcagttctacacttgagcaatctcctcaatagctaaagcttccacaatgcagattttgtgatgacaaattcagcatcacccagcagaacttaggtttttttctgtcctccgtttcctgacctttttcttctgagtgctttatgtcacctcgtgaaccatcctttccttagtcatctacctagcagtcctgattcttttgacttgtctccctacaccacaataaatcactaattactatggattcaatccctaaaatttgcacaaacttgcaaatagattacgggttgaaacttagagatttcaaacttgagaaaaaagtttaaatcaagaaaaatgacctttaccttgagagtagaggcaatgtcatttccaggaataattataataatattgtgtttaatatttgtatgtaacatttgaataccttcaatgttcttatttgtgttattttaatctcttgatgttactaactcatttggtagggaagaaaacatgctaaaataggcatgagtgtcttattaaatgtgacaagtgaatagatggcagaaggtggattcatattcagttttccatcaccctggaaatcatgcggagatgatttctgcttgcaaataaaactaacccaatgaggggaacagctgttcttaggtgaaaacaaaacaaacacgccaaaaacctttattctctttattatgaatcaaatttttcctctcagataattgttttatttatttatttttattattattgttattatgtccagtctcactctgtcgcctaagctggcatgattcgaatgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgactctggctaactagagaacccactgcttactggcttatcgaaattaatacgactcactatagggagacccaagcttggtaccgagctcggatcgaattcgatatcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgagctagcggatccgacctcgagggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcgggatccactagtaacggccgccagtgtgctgtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagatcgagcatgcatctagggcggccaattcttaaaactgggagtgggttgttcccactcactccacccatgcggtgttgtactctgttattacggtaactttgtacgccagtttttcccacccttccccataatgtaacttagaagtttgtacaatatgaccaataggtgacaatcatccagactgtcaaaggtcaagcacttctgtttccccggtcaatgaggatatgctttacccaaggcaaaaaccttagagatcgttatccccacactgcctacacagagcccagtaccatttttgatataattgggttggtcgctccctgcaaacccagcagtagacctggcagatgaggctggacattccccactggcgacagtggtccagcctgcgtggctgcctgctcacccttcttgggtgagaagcctaattattgacaaggtgtgaagagccgcgtgtgctcagtgtgcttcctccggcccctgaatgtggctaaccttaaccctgcagccgttgcccataatccaatgggtttgcggtcgtaatgcgtaagtgcgggatgggaccaactactttgggtgtccgtgtttcctgtttttcttttgattgcattttatggtgacaatttatagtgtatagattgtcatcatgccccgggataattcctgcagccaatatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaggggttaattaaagccaggcatgtgatgtacacctgtagtcccagctactcaggaggccgaaggaaactggagaaactgggaggagtatccagatgtcctgtccctgtaagggatattatcctggacaacatagcaagacctcgtctctacttaaaaaaaaagccaggtgtggtggcatgtgcctgtagtcctacctactcgggaggctgaggaatgtgggaggtggaggttgcagtgacctgagatcgtgccactgcactcaagaccagcctgatcaacatggtgaaaccctgtctctactaaaaatacaagctggacgtggtggcacgtgcctgtagtcccagctactcgggaggctgaggaacccaggaggtggaggttgcagtgagctgagatcgcgccactgcactcgagaccatcctgaccaatatggtgaaaccccatctctactaaagatacaaaaatagtgcagcaatgaacatgagagtgcaactatctcttcaatgtactgggtgtgggtggagagaaccgcttgtgagcttgtctctgcggccagctgagaacacaggcctcaccaagcaggaaaagccacctctgccctaaaagtaagagactcctgggcgtggagcctaccctggggtatggttgataaacacagtgtgaaaacggcagttgggcactgcactgcccggtgatggtgccacggtggctgctgggcgtggtggtgcctgcctgtaatcccagctactcagaaggctcaggaaccagggagtcggtggctagagtgagccgagattgcatcactgcactctattcaggtaggtgtagggtggggggtgttgaggtttaagtaagcaaagtagcaggtgggggcacttctccctctaacactctcccctgttgaagctcttaagccaaggaggaggaggggggtgaggtgaaagatgagctggaggaccgcgctgggcatggtggcacacgcctataatcccagctactcaggaggctgaggaatccgggaggcagaggtcacagtgagccaagatcacgccactgtactcaagaccagcctggccaacatggtgaaatcccgtctctattaaaaatacaagtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 8 - DNA, 1172 bp, Artificial Sequence
  synthetic
tctagagcagcaccatggcctgaaataacctctgaaagaggaacttggttaggtaccttctgaggcggaaagaaccagctgtggaatgtgtgtcagttagggtggttaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccgtggcggaatctgagttgcagctggtggcgcagcgcatccgcagtttccccgacttccccatccccggcgtgctgtttagggatatctcgcccctcctgaaggaccccgcctccttccgagcttccatccgcctcctggccagtcaccttaagtccacgcatggcggcaagatcgactacatcgcaggcctagactccaggggattcttgtttggcccctccctagctcaggagctgggcctgggctgtgtgctcatccggaagcgagggaagctgccaggccccacagtgtcagcctcctatgctctcgagtatggcaaggctgaactagaaatccagaaagacgccttagaacctggccagaaagtggttgttgtagatgatctcctggccactggaggaaccatgtgcgctgcctgtgagctgctgggccagctacaggctgaggtggtggagtgtgtgagcctggtggagctgacctcacttaagggcagagagaagctaggatcagtaccattcttctctctcctgcaatatgagtgacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtctaga

SEQ ID NO: 9 - DNA, 11872 bp, Artificial Sequence
  synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgaaggtgggtggatcacccgaggtcaggagttcaagaccagcctggccaacatggtaaaacctcgtctctactaaaaaatacgaaaaattagctggttgtggtggtgcgtgcttgtaatcccagctactcgggaggctgaggcaggagaatcacttgaatctgggaggcagaggttgcagtgagctgagatagtgccattgcactccagcctgggcaacagacggagactctgtctccaaaaaaaaaaaaaaaaatcttagaggacaagaatggctctctcaaacttttgaagaaagaataaataaattatgcagttctagaagaagtaatggggatataggtgcagctcatgatgaggaagacttagcttaactttcataatgcatctgtctggcctaagacgtggtgagctttttatgtctgaaaacattccaatatagaatgataataataatcacttctgaccccccttttttttcctctccctagactgtgaagcagaaaccccatatttttcttagggaagtggctacgcactttgtatttatattaacaactaccttatcaggaaattcatattgttgcccttttatggatggggaaactggacaagtgacagagcaaaatccaaacacagctggggatttccctcttttagatgatgattttaaaagaatgctgccagagagattcttgcagtgttggaggacatatatgacctttaagatattttccagctcagagatgctatgaatgtatcctgagtgcatggatggacctcagttttgcagattctgtagcttatacaatttggtggttttctttagaagaaaataacacatttataaatattaaaataggcccaagaccttacaagggcattcatacaaatgagaggctctgaagtttgagtttgttcactttctagttaattatctcctgcctgtttgtcataaatgcgtttagtagggagctgctaatgacaggttcctccaacagagtgtggaagaaggagatgacagctggcttcccctctgggacagcctcagagctagtggggaaactatgttagcagagtgatgcagtgaccaagaaaatagcactaggagaaagctggtccatgagcagctggtgagaaaaggggtggtaatcatgtatgccctttcctgttttattttttattgggtttccttttgcctctcaattccttctgacaatacaaaatgttggttggaacatggagcacctggaagtctggttcattttctctcagtctcttgatgttctctcgggttcactgcctattgttctcagttctacacttgagcaatctcctcaatagctaaagcttccacaatgcagattttgtgatgacaaattcagcatcacccagcagaacttaggtttttttctgtcctccgtttcctgacctttttcttctgagtgctttatgtcacctcgtgaaccatcctttccttagtcatctacctagcagtcctgattcttttgacttgtctccctacaccacaataaatcactaattactatggattcaatccctaaaatttgcacaaacttgcaaatagattacgggttgaaacttagagatttcaaacttgagaaaaaagtttaaatcaagaaaaatgacctttaccttgagagtagaggcaatgtcatttccaggaataattataataatattgtgtttaatatttgtatgtaacatttgaataccttcaatgttcttatttgtgttattttaatctcttgatgttactaactcatttggtagggaagaaaacatgctaaaataggcatgagtgtcttattaaatgtgacaagtgaatagatggcagaaggtggattcatattcagttttccatcaccctggaaatcatgcggagatgatttctgcttgcaaataaaactaacccaatgaggggaacagctgttcttaggtgaaaacaaaacaaacacgccaaaaacctttattctctttattatgaatcaaatttttcctctcagataattgttttatttatttatttttattattattgttattatgtccagtctcactctgtcgcctaagctggcatgattcgaatgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgactctggctaactagagaacccactgcttactggcttatcgaaattaatacgactcactatagggagacccaagcttggtaccgagctcggatcgaattcgatatcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgagctagcggatccgacctcgagggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcgggatccactagtaacggccgccagtgtgctgtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagatcgagcatgcatctagggcggccaattcttaaaactgggagtgggttgttcccactcactccacccatgcggtgttgtactctgttattacggtaactttgtacgccagtttttcccacccttccccataatgtaacttagaagtttgtacaatatgaccaataggtgacaatcatccagactgtcaaaggtcaagcacttctgtttccccggtcaatgaggatatgctttacccaaggcaaaaaccttagagatcgttatccccacactgcctacacagagcccagtaccatttttgatataattgggttggtcgctccctgcaaacccagcagtagacctggcagatgaggctggacattccccactggcgacagtggtccagcctgcgtggctgcctgctcacccttcttgggtgagaagcctaattattgacaaggtgtgaagagccgcgtgtgctcagtgtgcttcctccggcccctgaatgtggctaaccttaaccctgcagccgttgcccataatccaatgggtttgcggtcgtaatgcgtaagtgcgggatgggaccaactactttgggtgtccgtgtttcctgtttttcttttgattgcattttatggtgacaatttatagtgtatagattgtcatcatgccccgggataattcctgcagccaatatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaggggttaattaaagccaggcatgtgatgtacacctgtagtcccagctactcaggaggccgaaggaaactggagaaactgggaggagtatccagatgtcctgtccctgtaagggatattatcctggacaacatagcaagacctcgtctctacttaaaaaaaaagccaggtgtggtggcatgtgcctgtagtcctacctactcgggaggctgaggaatgtgggaggtggaggttgcagtgacctgagatcgtgccactgcactcaagaccagcctgatcaacatggtgaaaccctgtctctactaaaaatacaagctggacgtggtggcacgtgcctgtagtcccagctactcgggaggctgaggaacccaggaggtggaggttgcagtgagctgagatcgcgccactgcactcgagaccatcctgaccaatatggtgaaaccccatctctactaaagatacaaaaatagtgcagcaatgaacatgagagtgcaactatctcttcaatgtactgggtgtgggtggagagaaccgcttgtgagcttgtctctgcggccagctgagaacacaggcctcaccaagcaggaaaagccacctctgccctaaaagtaagagactcctgggcgtggagcctaccctggggtatggttgataaacacagtgtgaaaacggcagttgggcactgcactgcccggtgatggtgccacggtggctgctgggcgtggtggtgcctgcctgtaatcccagctactcagaaggctcaggaaccagggagtcggtggctagagtgagccgagattgcatcactgcactctattcaggtaggtgtagggtggggggtgttgaggtttaagtaagcaaagtagcaggtgggggcacttctccctctaacactctcccctgttgaagctcttaagccaaggaggaggaggggggtgaggtgaaagatgagctggaggaccgcgctgggcatggtggcacacgcctataatcccagctactcaggaggctgaggaatccgggaggcagaggtcacagtgagccaagatcacgccactgtactcaagaccagcctggccaacatggtgaaatcccgtctctattaaaaatacaatctagagcagcaccatggcctgaaataacctctgaaagaggaacttggttaggtaccttctgaggcggaaagaaccagctgtggaatgtgtgtcagttagggtggttaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccgtggcggaatctgagttgcagctggtggcgcagcgcatccgcagtttccccgacttccccatccccggcgtgctgtttagggatatctcgcccctcctgaaggaccccgcctccttccgagcttccatccgcctcctggccagtcaccttaagtccacgcatggcggcaagatcgactacatcgcaggcctagactccaggggattcttgtttggcccctccctagctcaggagctgggcctgggctgtgtgctcatccggaagcgagggaagctgccaggccccacagtgtcagcctcctatgctctcgagtatggcaaggctgaactagaaatccagaaagacgccttagaacctggccagaaagtggttgttgtagatgatctcctggccactggaggaaccatgtgcgctgcctgtgagctgctgggccagctacaggctgaggtggtggagtgtgtgagcctggtggagctgacctcacttaagggcagagagaagctaggatcagtaccattcttctctctcctgcaatatgagtgacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtctagagtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 10 - DNA, 1479 bp, Artificial Sequence
  synthetic
gccgccaccatggcctcctccctgtactccttcctgctggccctgtccatcgtgtacatcttcgtggcccccacccactccgaccaggagtcctgcaagggcagatgtacagagggctttaacgtggataagaagtgccagtgcgacgagctgtgctcctactaccagtcctgctgcacagactacacagccgagtgtaagccccaggtgacaagaggcgacgtgttcaccatgcccgaggacgagtacacagtgtacgacgacggcgaggagaagaacaacgccaccgtgcacgagcaggtgggcggccctagcctgacctccgatctgcaggcccagagcaagggcaaccctgagcagacccccgtgctgaagcctgaggaggaggcccctgcccccgaggttggagcttccaagcccgagggcatcgattccagacctgagaccctgcaccccggcaggcctcagcctccagctgaggaggagctgtgcagcggcaagcccttcgacgcctttacagacctgaagaacggctccctgttcgcctttagaggccagtactgttacgagctggatgagaaggccgtgaggcctggctaccctaagctgatcagagatgtgtggggcatcgagggccccatcgacgccgcttttaccagaatcaattgtcagggcaagacctacctgtttaagggctcccagtactggaggtttgaggacggcgtgctggaccctgactaccctagaaacatctccgatggcttcgacggcatccctgataacgtggacgccgccctggccctgcccgctcacagctattccggcagggagagagtgtacttctttaagggcaagcagtactgggagtaccagttccagcaccagcccagccaggaggagtgcgagggcagcagcctgtccgccgtgtttgagcacttcgccatgatgcagagggatagctgggaggatatcttcgagctgctgttctggggcagaacctccgccggcaccagacagcctcagtttatctccagagactggcacggcgtgcccggccaggtggatgctgctatggccggcagaatctacatctccggcatggcccctaggccttccctggccaagaagcagaggttcagacacaggaacagaaagggctacaggtcccagagaggccactccaggggcagaaaccagaatagcagaagaccttccagggccacctggctgagcctgttctccagcgaggagagcaatctgggcgccaacaactacgatgattacagaatggattggctggtgcccgccacctgcgagcccatccagtccgtgttcttcttctccggcgataagtactacagggtgaacctgaggacaaggagagtggataccgtggaccctccctaccccagaagcatcgcccagtactggctgggctgtcccgcccctggccacctgcaccaccaccaccatcactga